(19) United States
(12) Patent Application Publication
(10) Pub. No.: US 2013/0096443 A1
(43) Pub. Date: Apr. 18, 2013

(54) PUSH-TYPE DISCHARGE ASSEMBLY OF BLOOD PRESSURE MONITOR

(71) Applicants: Chao-Man Tseng, New Taipei City (TW); Lien-Sheng Cehn, New Taipei City (TW); Yung-Ming Tsai, Taipei City (TW)

(72) Inventors: Chao-Man Tseng, New Taipei City (TW); Lien-Sheng Cehn, New Taipei City (TW); Yung-Ming Tsai, Taipei City (TW)

(73) Assignee: K-Jump Health Co., Ltd.

(21) Appl. No.: 13/273,169

(22) Filed: Oct. 13, 2011

(51) Int. Cl. A61B 5/02

(52) U.S. Cl. 600/485

(57) ABSTRACT

A push-type discharge assembly of a blood pressure monitor includes a valve base, a spring, a driven element, a restricting element, and a pressing element. The valve base is provided with an accommodating trough and an intake pipe. The spring is disposed in the accommodating trough. The driven element abuts against one end of the spring. The driven element is formed with a protrusion. The restricting element covers the accommodating trough. The restricting element has a central through-hole and is formed with a restricting notch. The pressing element passes through the central through-hole. The pressing element has a sealing body and a driving portion disposed on a lower edge of the sealing body to drive the driven element. When the pressing element is pressed downwards, the sealing body moves downwards to form a discharge channel between the pressing element and the central through-hole.

### BACKGROUND OF THE INVENTION

1. Field of the Invention

The present invention relates to a blood pressure monitor, and in particular to a push-type discharge assembly of a blood pressure monitor.

2. Description of Prior Art

A blood pressure monitor is an instrument for measuring the blood pressure of a human body, in which an inflatable blade is provided. After the inflatable blade is inflated, the inflated blade presses the vessels of a user, so that the blood pressure of the user can be measured. When the measurement of the blood pressure is completed, the air in the inflated blade has to be discharged for subsequent use.

In the conventional blood pressure monitor, the discharging process is achieved by loosening a knob manually, whereby the air in the inflatable blade can be discharged. After the discharging process is completed, the user has to tighten the knob, which makes this operation not so convenient. In addition to the above-mentioned blood pressure monitor, an electronic blood pressure monitor is developed, in which a small motor is provided to drive a discharge valve for discharging the air in the inflatable blade. However, such an electronic blood pressure monitor has more components and higher price.

Therefore, it is a prospective issue for the present Inventor to solve the above problems.

### SUMMARY OF THE INVENTION

The present invention is to provide a push-type discharge assembly of a blood pressure monitor, whereby the discharging process is achieved by one push action.

The present invention provides a push-type discharge assembly of a blood pressure monitor, including:

a valve base provided with an accommodating trough and an intake pipe in communication with the accommodating trough;

a spring disposed in the accommodating trough;

a driven element abutting against one end of the spring, the driven element being formed with at least one protrusion;

a restricting element provided in the accommodating trough, the restricting element having a central through-hole, a periphery of the restricting element being formed with a restricting notch for allowing the protrusion to be inserted therein; and

a pressing element passing through the central through-hole, the pressing element having a sealing body and a driving portion disposed on a lower edge of the sealing body to drive the driven element,

wherein the sealing body moves downwards to form a discharge channel between the pressing element and the central through-hole when the pressing element is pressed downwards.

In comparison with prior art, the present invention has advantageous features as follows.

According to the present invention, the sealing body is put around the pressing element, and the pressing element has a driving portion disposed on a lower edge of the sealing body to drive the driven element. Thus, the user only needs to press the pressing element downwards by his/her finger to thereby cause the driving portion of the pressing element to move downwards. As a result, the sealing body is moved downwards to form a discharge channel between the pressing element and the central through-hole. Therefore, the discharging process can be achieved by one push action, which is very convenient for the user. Further, it is not necessary to mount a small motor in the present invention, so that the cost and price of the present invention are reduced.

### BRIEF DESCRIPTION OF DRAWING

FIG. 1 is an exploded perspective view of the present invention;

FIG. 2 is an assembled cross-sectional view of the present invention;

FIG. 3 is an assembled cross-sectional view of the present invention;

FIG. 4 is a schematic view showing that the pressing element of the present invention is pressed for the first time;

FIG. 5 is a schematic view showing that the driving portion of the present invention is driven to rotate;

FIG. 6 is a schematic view showing that the protrusion of the present invention enters the restricting notch;

FIG. 7 is a cross-sectional view of FIG. 6, showing that the sealing body moves downwards for discharge;

FIG. 8 is a schematic view showing that the pressing element of the present invention is pressed for the second time;

FIG. 9 is a schematic view showing that the driving portion of the present invention is driven to rotate;

FIG. 10 is a schematic view showing that the pressing element of the present invention moves upwards;

FIG. 11 is a cross-sectional view of FIG. 10, showing that the sealing body is to move upwards for sealing; and

FIG. 12 is an assembled cross-sectional view showing another embodiment of the present invention.

### DETAILED DESCRIPTION OF THE INVENTION

The detailed description and technical contents of the present invention will become apparent with the following detailed description accompanied with related drawings. It is noteworthy to point out that the drawings is provided for the illustration purpose only, but not intended for limiting the scope of the present invention.

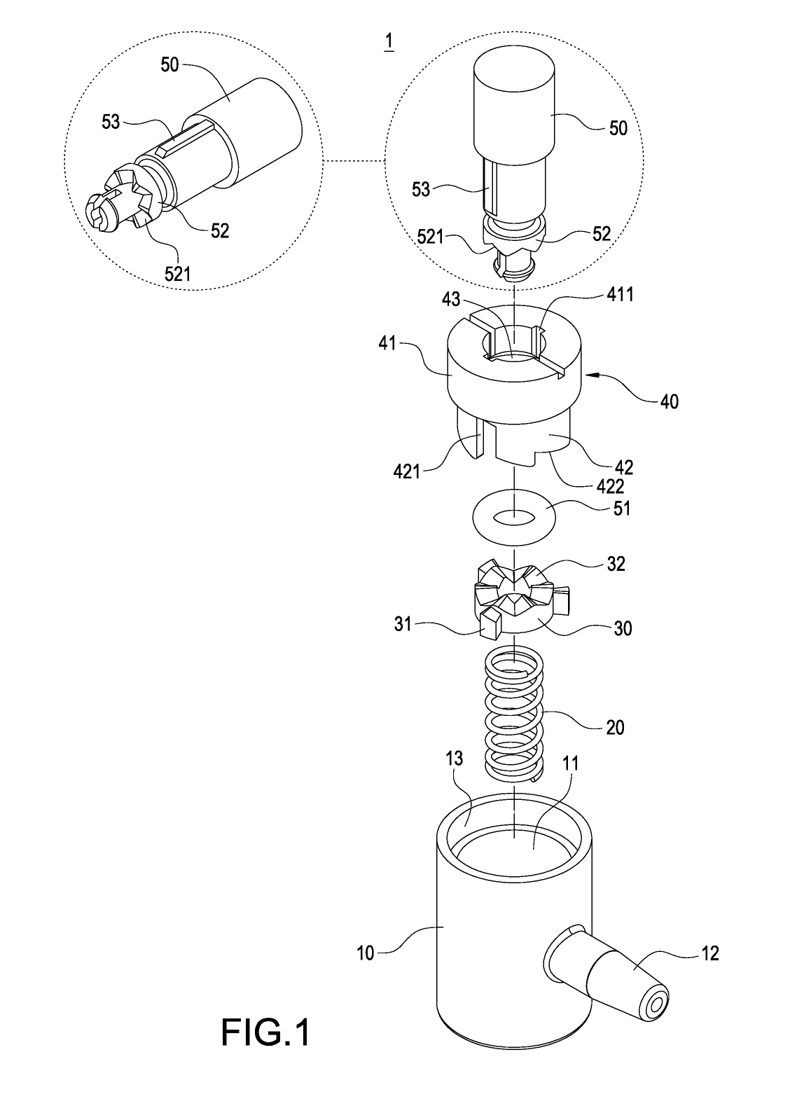

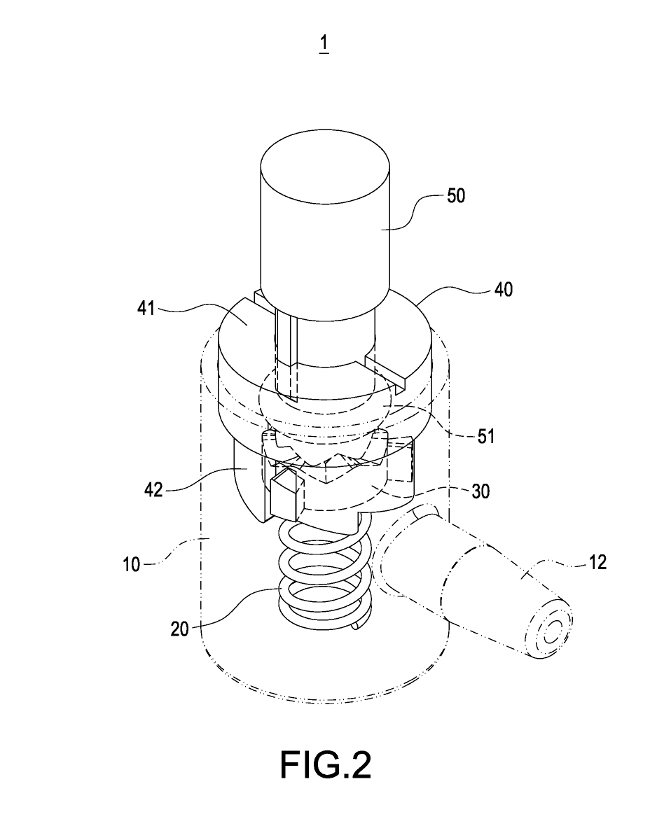

Please refer to FIGS. 1 to 3. The present invention provides a push-type discharge assembly 1 of a blood pressure monitor, which includes a valve base 10, a spring 20, a driven element 30, a restricting element 40, and a pressing element 50.

The valve base 10 is substantially formed into a cylindrical shape. The valve base 10 has an accommodating trough 11 and an intake pipe 12 in communication with the accommodating trough 11. One end of the accommodating trough 11 is formed with an opening for allowing other components to be accommodated in the valve base 10. The inner upper wall of the valve base 10 is formed with an annular groove 13 for allowing a portion of the restricting element 40 to be inserted therein.

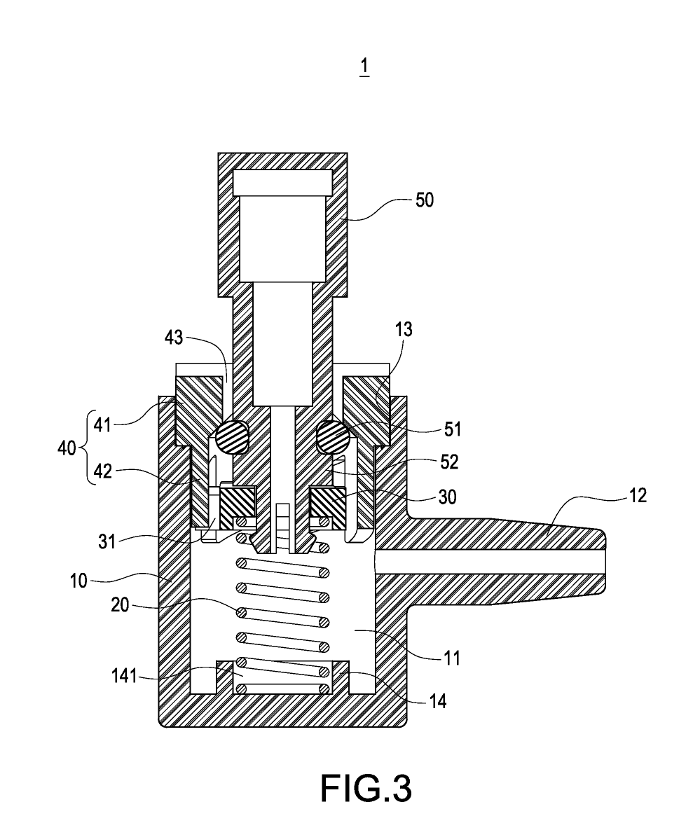

The spring 20 is disposed in the accommodating trough 11. In order to make the spring 20 to be positioned in the accommodating trough 11 accurately, as shown in FIG. 3, the inner bottom wall of the valve base 10 is formed with a flange 14. The inner edge of the flange 14 encloses a positioning hole 141 for positioning the spring 20.

The driven element 30 abuts against one end (free end) of the spring 20. The periphery of the driven element 30 is formed with at least one protrusion 31 (three in the present embodiment). The upper surface of the driven element 30 is formed with a waved surface 32.

The restricting element 40 covers the accommodating trough 11 in a sealing manner. More specifically, the restricting element 40 is constituted of a large diameter section 41 and a small diameter section 42. The outer diameter of the large diameter section 41 is equal to the inner diameter of the annular groove 13 formed on the inner wall of the valve base 10. The outer diameter of the small diameter section 42 is equal to the inner diameter of the valve base 10. By this structure, the restricting element 40 can be inserted into an upper portion of the valve base 10. As shown in FIG. 1, the inner wall of the large diameter section 41 is provided with two longitudinal slots 411.

The restricting element 40 has a central through-hole 43 penetrating the large diameter section 41 and the small diameter section 42. The periphery of the restricting element 40 (on the small diameter section 42) is formed with a restricting notch 421 for allowing the protrusion 31 to be inserted therein. Since the driven element 30 has three protrusions 31, one of the protrusions 31 enters the restricting notch 421 once the driven element 30 rotates 120 degrees.

The pressing element 50 is substantially formed into a cylindrical shape and passes through the central through-hole 43. A sealing body 51 is put around the pressing element 50. The sealing body 51 is used to control the entrance and discharge of air, so that any suitable element capable of achieving an airtight effect will be embraced by the scope of the present invention. At the lower edge of the sealing body 51, the lower portion of the pressing element 50 is provided with a driving portion 52 for driving the driven element 30. The surface of the driving portion 52 is also formed with a waved surface 521. The waved surfaces 32 and 521 of the driven element 30 and the driving portion 52 are configured to increase the friction force therebetween. The peripheral surface of the pressing element 50 is formed with an engaging key 53 to be engaged with the longitudinal slot 411 of the restricting element 40, whereby the pressing element 50 cannot rotate relative to the restricting element 40 but can only move up and down relative to the restricting element 40.

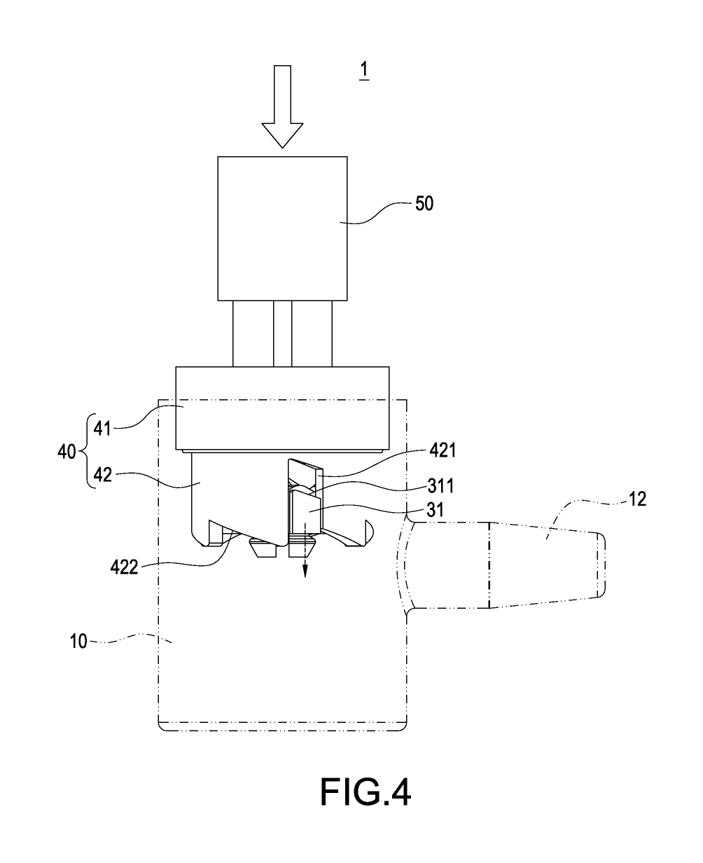

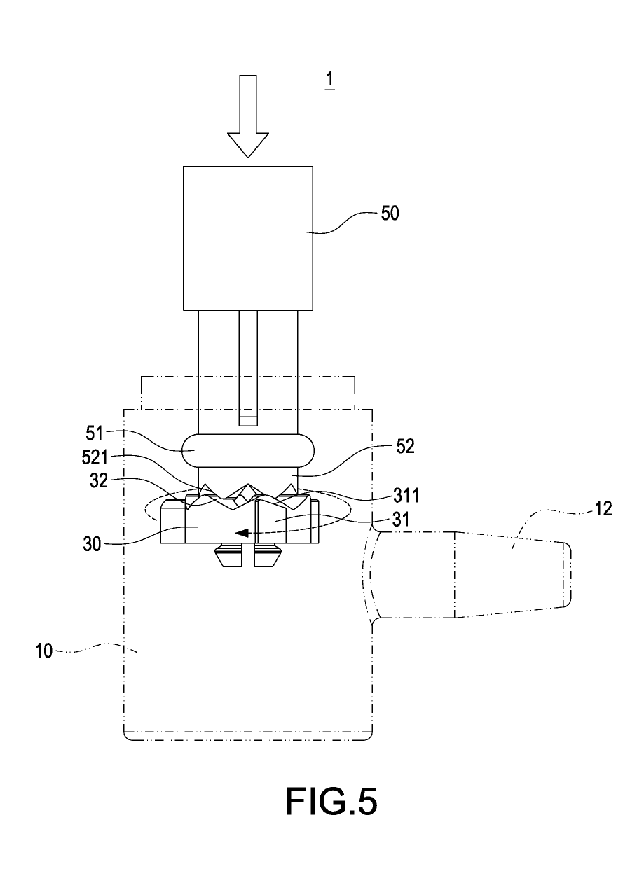

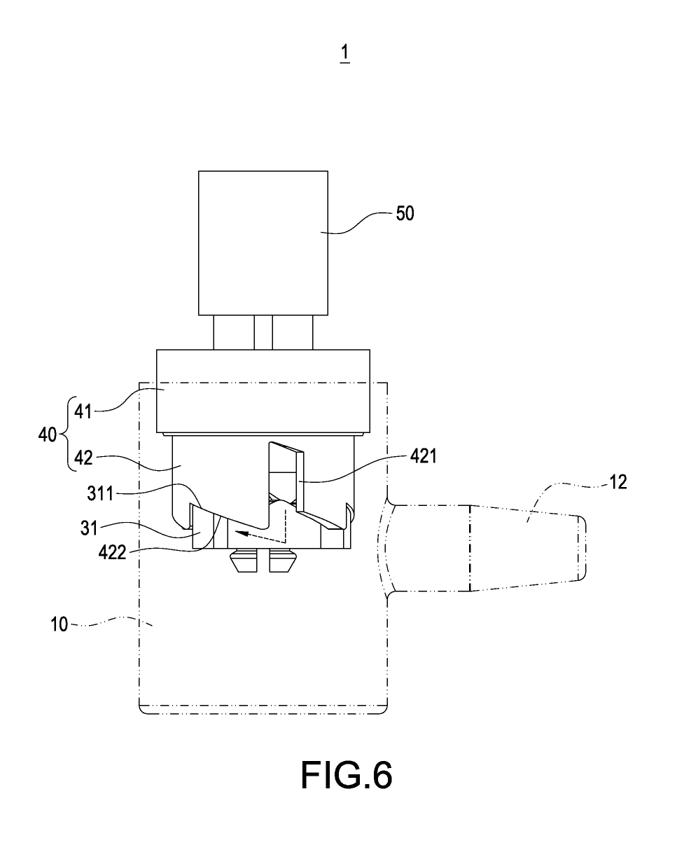

Please refer to FIGS. 4 to 7. The operation and effect of the present invention when the pressing element 50 is pressed for the first time will be described. The state shown in FIG. 3 is defined as an original sealing state. In this state, the elastic force of the spring 20 urges the driven element 30 to move upwards to raise the pressing element 50. As a result, the sealing body 51 seals the gap between the pressing element 50 and the central through-hole 43.

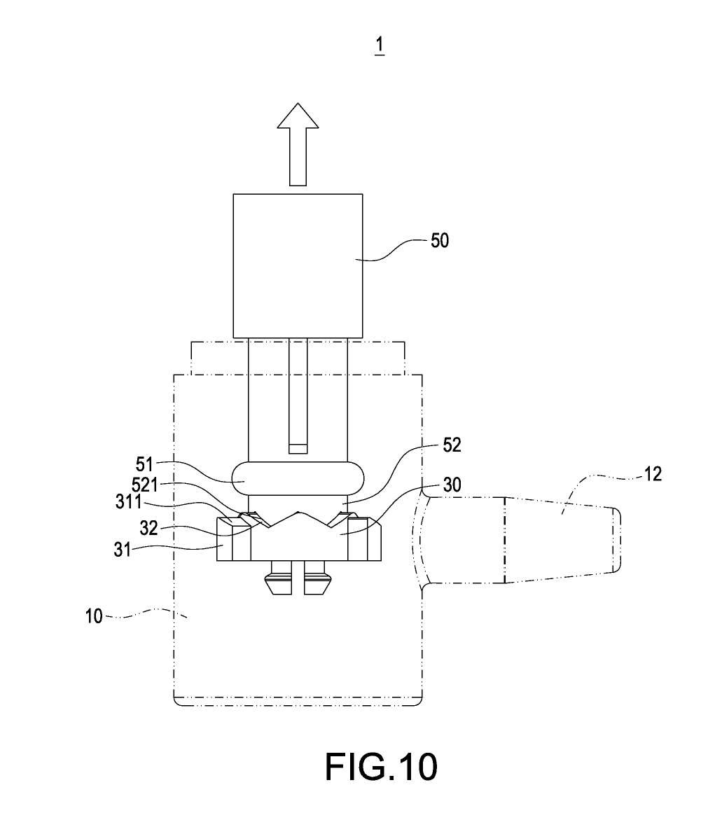

As shown in FIG. 4, when the user presses the pressing element 50 for the first time, the driving portion 52 of the pressing element 50 is moved downwards to urge the driven portion 30 to move downwards. At this time, the protrusion 31 on the periphery of the driven element 30 moves downwards from the restricting notch 421 of the restricting element 40 to leave the restricting notch 421. It should be noted that the top surface of each protrusion 31 is formed with a first guiding slope 311, and the lower edge of the small diameter section 42 of the restricting element 40 is formed with a plurality of second guiding slopes 422 at intervals. Further, the contact surfaces between the driving portion 52 and the driven element 30 are formed into waved surfaces to facilitate the driven element 30 to generate rotation of a few degrees (because the pressing element 50 is fixed by the longitudinal slots 411 and thus unable to rotate relative to the restricting element 40). By this structure, as shown in FIG. 4, when the protrusion 31 moves downwards in the direction of the arrow, the inclination direction of the first guiding slope 311 is equal to that of the second guiding slope 422. Thus, the protrusion 31 of the driven element 30 can rotate a few degrees and then slides on the second guiding slope 422 from left to right. Finally, the protrusion 31 enters the restricting notch 421, which causes the driven element 20 to move upwards to urge the pressing element 50 to move upwards (FIG. 10).

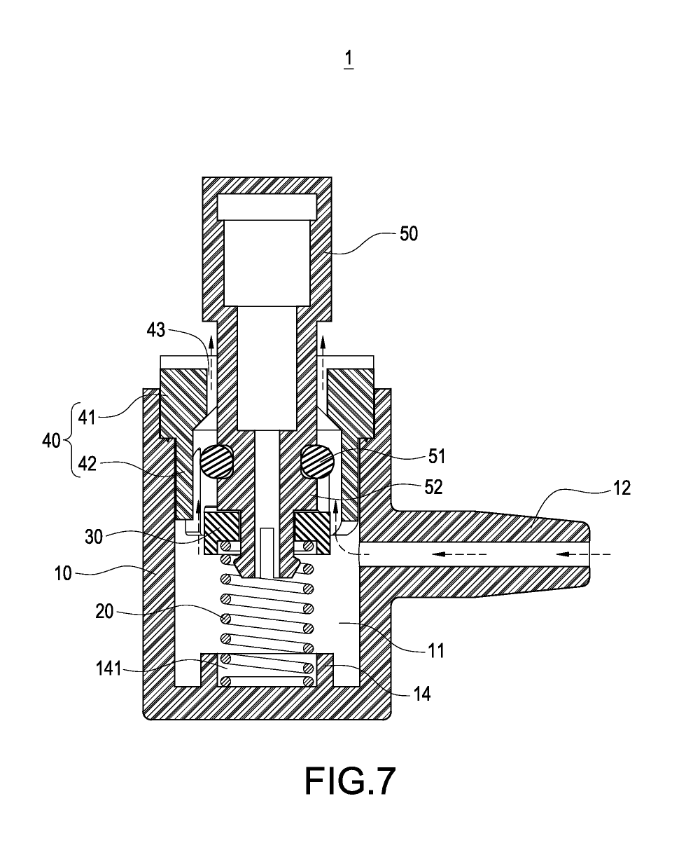

As shown in FIG. 7, since the pressing element 50 is pressed, the sealing body 51 is moved downwards to generate a discharge channel between the pressing element 50 and the central through-hole 43. In this way, the air in the intake pipe 12 can be discharged to the outside via the discharge channel.

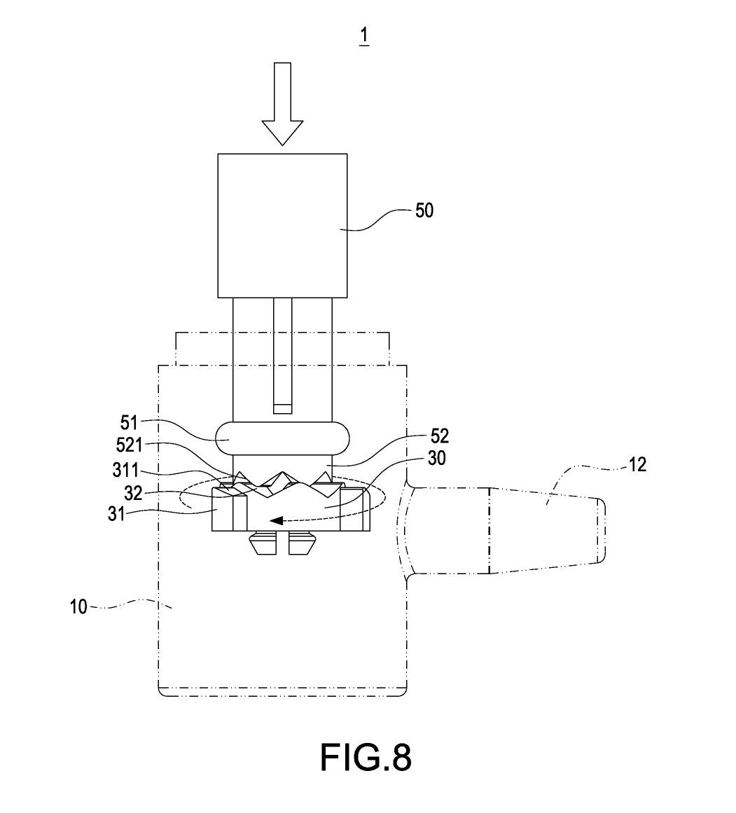

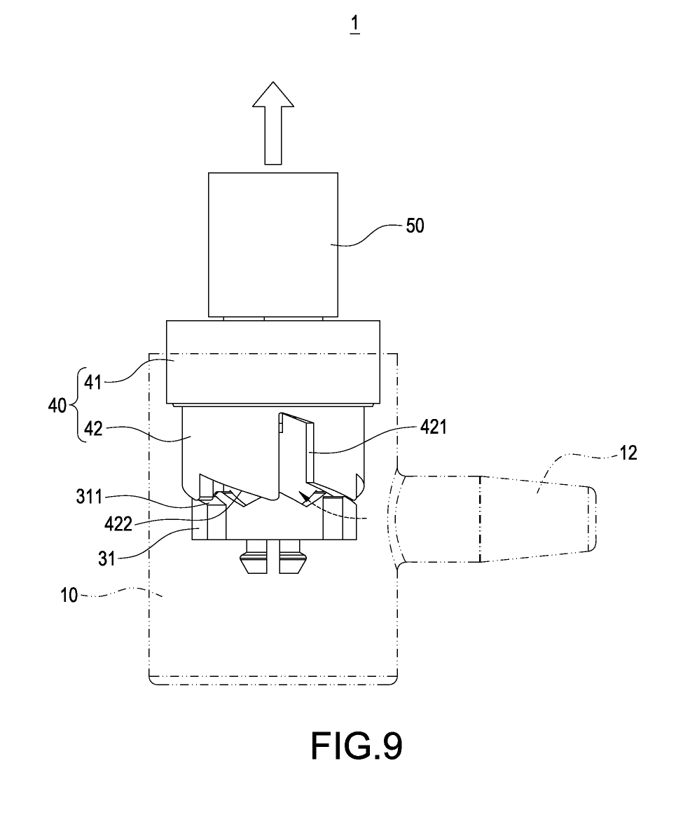

Please refer to FIGS. 8 to 11. The operation and effect of the present invention when the pressing element 50 is pressed for the second time will be described as follows. Starting from the state shown in FIG. 7, when the pressing element 50 is pressed, as shown in FIG. 8, the waved surfaces 521 and 32 between the driving portion 52 and the driven element 30 facilitate the driven element 30 to rotate a few degrees. Thus, after the protrusion 31 of the driven element 30 rotates a few degrees, the protrusion 31 slides on the second guiding slope 422 from left to right. Finally, the protrusion 31 enters the restricting notch 421, which causes the driven element 30 to move upwards to urge the pressing element 50 to move upwards (FIG. 10).

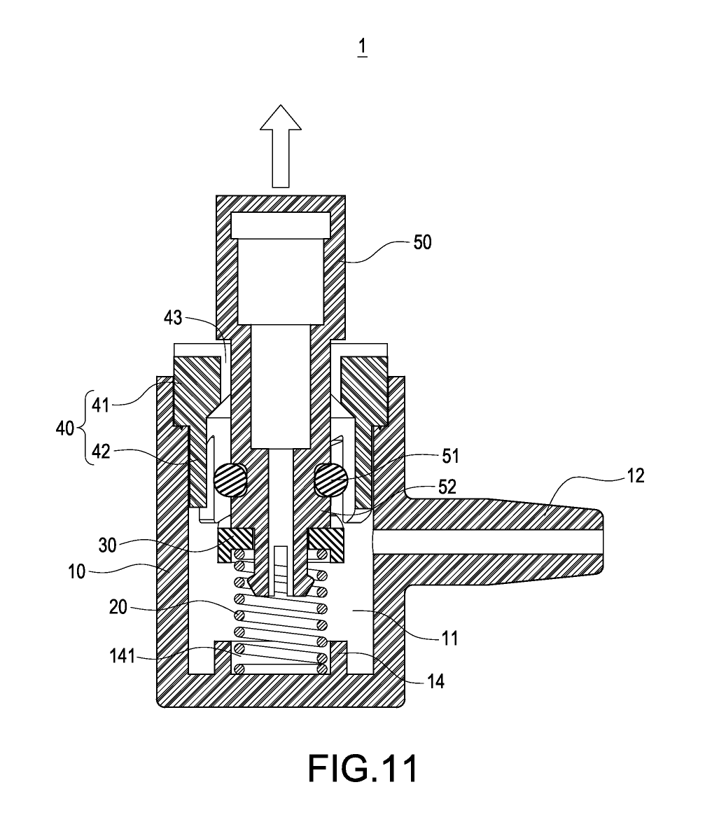

As shown in FIG. 11, when the pressing element 50 moves upwards, the sealing body 51 also moves upwards to seal the discharge channel between the pressing element 50 and the central through-hole 43, thereby returning to the original sealing state shown in FIG. 3.

Therefore, the present invention employs a two-stage operation. In other words, the user presses the pressing element 50 for the first time to discharge air, and the user presses the pressing element 50 for the second time to seal. Thus, it is very convenient for use.

In comparison with prior art, the present invention has advantageous features as follows.

According to the present invention, the sealing body 51 is put around the pressing element 50, and the pressing element 50 has a driving portion 52 disposed on a lower edge of the sealing body 51 to drive the driven element 30. Thus, the user only needs to press the pressing element 50 downwards by his/her finger to thereby cause the driving portion 52 of the pressing element 50 to move downwards. As a result, the sealing body 51 is moved downwards to form a discharge channel between the pressing element 50 and the central through-hole 43. Therefore, the discharging process can be achieved by one push action, which is very convenient for the user. Further, it is not necessary to mount a small motor in the present invention, so that the cost and price of the present invention are reduced.

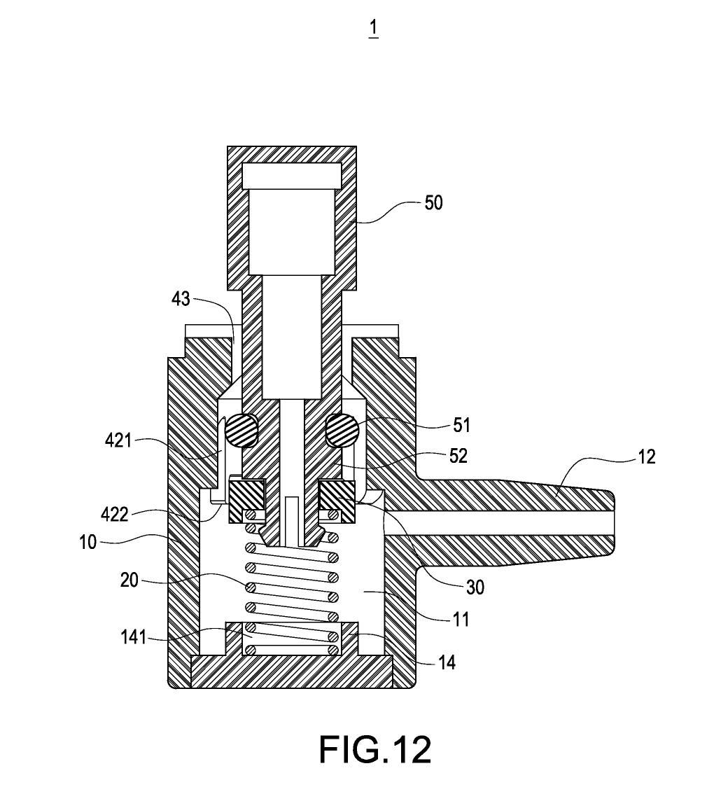

Please refer to FIG. 12, which shows another embodiment of the present invention. The difference between the present invention and the previous embodiment lies in that: the restricting notch 421 of the restricting element 40 and the second guiding slopes 422 are directly formed in the valve base 10 to form one body. By this structure, the present embodiment also achieves the same effect as the previous embodiment.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A push-type discharge assembly of a blood pressure monitor, including:
a valve base provided with an accommodating trough and an intake pipe in communication with the accommodating trough;
a spring disposed in the accommodating trough;
a driven element abutting against one end of the spring, the driven element being formed with at least one protrusion;
a restricting element provided in the accommodating trough, the restricting element having a central through-hole, a periphery of the restricting element being formed with a restricting notch for allowing the protrusion to be inserted therein; and
a pressing element passing through the central through-hole, the pressing element having a sealing body and a driving portion disposed on a lower edge of the sealing body to drive the driven element,
wherein the sealing body moves downwards to form a discharge channel between the pressing element and the central through-hole when the pressing element is pressed downwards.

2. The push-type discharge assembly of a blood pressure monitor according to claim 1, wherein an upper surface of the driven element is formed with a waved surface, a surface of the driving portion is also formed with a waved surface, the engagement between the two waved surfaces causes the driven element to rotate a few degrees relative to the driving portion.

3. The push-type discharge assembly of a blood pressure monitor according to claim 2, wherein an inner upper wall of the valve base is formed with an annular groove, the restricting element comprises a large diameter section and a small diameter section, the large diameter section is engaged in the annular groove.

4. The push-type discharge assembly of a blood pressure monitor according to claim 3, wherein the inner wall of the large diameter section is provided with a longitudinal slot, a peripheral surface of the pressing element is formed with an engaging key engaged in the longitudinal slot.

5. The push-type discharge assembly of a blood pressure monitor according to claim 4, wherein a top surface of the protrusion is formed with a first guiding slope, a lower edge of the small diameter section is formed with a plurality of second guiding slopes at intervals, the inclination direction of the first guiding slope is equal to that of the second guiding slope, so that the protrusion is capable of rotating a few degrees and then sliding on the second guiding slope.

6. The push-type discharge assembly of a blood pressure monitor according to claim 5, wherein an inner bottom wall of the valve base is formed with a flange, an inner periphery of the flange encloses a positioning hole for positioning the spring.

7. The push-type discharge assembly of a blood pressure monitor according to claim 1, wherein the restricting element covers the accommodating trough in a sealing manner.

8. The push-type discharge assembly of a blood pressure monitor according to claim 1, wherein the restricting notch is formed in the valve base.

9. The push-type discharge assembly of a blood pressure monitor according to claim 8, wherein a top surface of the protrusion is formed with a first guiding slope, the interior of the valve base is formed with a plurality of second guiding slopes at intervals, the inclination direction of the first guiding slope is equal to that of the second guiding slope, so that the protrusion is capable of rotating a few degrees and then sliding on the second guiding slope.

10. The push-type discharge assembly of a blood pressure monitor according to claim 1, wherein the sealing body is put around the pressing element.
